(19) United States
(12) Patent Application Publication
(10) Pub. No.: US 2009/0088660 A1
(43) Pub. Date: Apr. 2, 2009

(54) SYSTEM AND METHODS FOR NERVE RESPONSE MAPPING

(76) Inventor: Gerald McMorrow, Redmond, WA (US)

Correspondence Address: BLACK LOWE & GRAHAM, PLLC, 701 FIFTH AVENUE, SUITE 4800, SEATTLE, WA, 98104, US

(21) Appl. No.: 12/200,829

(22) Filed: Aug. 28, 2008

Related U.S. Application Data
(60) Provisional application No. 60/968,823, filed on Aug. 29, 2007.

(51) Int. Cl. A61B 5/0488

(52) U.S. Cl. 600/546

(57) ABSTRACT

Systems and methods are described to detect and anatomically map the location of active nerves or nerve bundles before, during, or after surgical procedures. The optical-based system supports and provides for methods to stimulate nerves in a non-electrolytic manner to activate or stimulate nerves within muscular tissue within the surgical field so that a given nerve's anatomical location or level of functional activity may be made contemporaneously apparent to the surgeon or operating personnel in the form of a neural activity map. The neural activity map provides for mapping of nerve location and classifying the mapped nerves by functional levels. The contemporaneously presented neural activity map enables the surgeon or operating personnel to initially plan a neural muscular surgery before undertaking incisions, or modify the surgical procedure based upon the updated neural mapping of the surgical region of interest.

### CROSS REFERENCE TO RELATED APPLICATIONS

The application claims priority to and incorporates by reference in its entirety U.S. Provisional Patent Application No. 60/968,823 filed Aug. 29, 2007.

### FIELD OF THE INVENTION

The invention relates generally to systems and procedures configured to implement neuromuscular surgeries.

### BACKGROUND OF THE INVENTION

Often surgeries are undertaken near nerve bundles or around neural muscular junctions in the vicinity of a nearby tumor. Attempts to surgically remove the tumor, especially when it is very close to the nerve bundles, may cause damage to nerves, especially those nerves that operate the respective innervated muscles. Nerve activity is measured by exposing the nerves present on muscular tissue with electrolyte solutions to stimulate nerves to cause muscle activity. The electrolytes may also cause the muscles to activate, thus providing a source of muscle noise that obscures the signal generated by the nerve upon exposure of the nerve to the same electrolyte. Accordingly, the measurement of how alive a nerve is, that is its functional activity, is made less certain as the source of muscular activity cannot be ascertained with high fidelity. That is, how much muscular activity is attributable to electrolyte stimulation of muscle, versus how much muscular activity is attributable to electrolyte stimulation of nerves that in turn stimulate the muscle cannot be accurately ascertained.

### SUMMARY OF THE PARTICULAR EMBODIMENTS

Systems and methods employing photonic activation of innervated muscular tissue are descried to assist the implementation of neuromuscular surgeries to enable a surgeon to conduct surgical procedures to minimize the risk of cutting live nerves, or to assess where to make incisions to maintain or maximize neural muscular functionality of an organ undergoing a surgical process.

The systems and methods include optical-based equipment and methods to detect and anatomically map the location of active nerves or nerve bundles before, during, or after surgical procedures. The optical-based system supports and provides for methods to stimulate nerves in a non-electrolytic manner to activate or stimulate nerves within muscular tissue within the surgical field so that a given nerve's anatomical location or level of functional activity may be made contemporaneously apparent to the surgeon or operating personnel in the form of a neural activity map. The neural activity map provides for mapping of nerve location and classifying the mapped nerves by functional levels. The contemporaneously presented neural activity map enables the surgeon or operating personnel to initially plan a neural muscular surgery before undertaking incisions, or modify the surgical procedure based upon the updated neural mapping of the surgical region of interest.

### BRIEF DESCRIPTION OF THE DRAWINGS

Embodiments for the system and are described in detail below with reference to the following drawings.

FIG. 1 depicts a functional block schematic of the Nerve Response Mapping system;

FIG. 2 depicts a perspective view of the optical scanner to stimulate nervous tissue;

FIG. 3 is a side view schematic illustration of the optical scanner;

FIG. 4 depicts an alternate functional block schematic embodiment of the Nerve Response Mapping system;

FIG. 5 depicts a scanning application of the Nerve Response Mapping system; and

FIG. 6 depicts a myleograph signal output mapped onto the image display of the surgical field depicted in FIG. 5.

### DETAILED DESCRIPTION OF THE PARTICULAR EMBODIMENTS

Particular include descriptions of optical-based systems and methods relating to the execution of surgeries upon nerve containing muscular tissue. The below described systems and methods enable the detection and anatomically mapping the location of active nerves or nerve bundles before, during, or after surgical procedures. The optical-based system supports and provides for methods to stimulate nerves in a non-electrolytic manner to activate or stimulate nerves within muscular tissue within the surgical field so that a given nerve's anatomical location or level of functional activity may be made contemporaneously apparent to the surgeon or operating personnel in the form of a neural activity map. The neural activity map provides for mapping of nerve location and classifying the mapped nerves by functional levels. The contemporaneously presented neural activity map enables the surgeon or operating personnel to initially plan a neural muscular surgery before undertaking incisions, or modify the surgical procedure based upon the updated neural mapping of the surgical region of interest. The systems and methods include employing photonic activation of innervated muscular tissue to assist the implementation of neuromuscular surgeries. Particular embodiments include optical based systems and methods to detect and anatomically map the location of active nerves or nerve bundles before, during, and/or after surgical procedures. The optical system supports and provides for methods to stimulate nerves without requiring the need for using electrolytes to activate or stimulate nerves residing on or within at least a portion of muscular tissue viewable in a surgical field so that nerve tissue presence and functional activity may be mapped to an anatomical location and displayed on an analog or digital image of the tissue regions susceptible to surgical procedures. The photo activation of nerves provides an improved signal to noise ratio to allow rapid, contemporaneous detection with improved nerve tissue mapping resolution. The contemporaneous detection, nerve activity estimation, and improved mapping resolution within the surgical field affords greater confidence to the operating physician in making decisions regarding anatomical locations for conducting surgery within the surgical field. That is, the optical-based systems and methods provides an improved way to enable a surgeon to conduct surgical procedures to either minimize the risk of cutting live nerves, and/or to assess where to make incisions in order to maintain and/or maximize neural muscular functionality of a given organ undergoing the surgical process. Other particular embodiments provide for optical based systems and methods to alert and guide the surgeon where to engage in the surgical procedures, including those involving tumor removal with preservation of neural muscular function of adjacent organs or structures within the surgical field. The optical based systems and methods phontonically activate at least one nerve or a nerve bundle or bundles to detect neural activity and anatomically map the location of active nerves or nerve bundles during surgical procedures. The photo activation of nerves provides an improved signal to noise ratio relative to electrolyte stimulation methods and enables contemporaneous detection with improved nerve tissue mapping resolution. The contemporaneous nerve mapping within the neural muscular region of interest provides for informing surgery decision makers as to the specific locations of nerves and their relative functional activity. The located nerves may be classified as to being active, partially active, and/or to be virtually inactive or non-functioning or dead, either as standalone nerve branches and/or nerve bundles. Yet other systems and methods are described acquiring, processing, and presenting anatomical maps of innervated tissue within a surgical field in which the nerve, nerves, or nerve bundles are stimulated by light. The effect of the light-stimulated nerve, nerves, or nerve bundle are measured by an electromyleograph that provides variations in signal output in proportion to the activity of a nerve or nerves, or the number of functional nerves undergoing photo stimulation capable of a generating muscular activity captured as electromyleograph signals (EMS). The EMS outputs are measured and associated with scanner light position within the scanner illuminated region or regions of interest (ROI). Signal maps are then constructed by overlaying EMS readings to locations within the ROI. The EMS signal maps may be overlaid on video or computer generated images of the surgical field or surgical ROI. Yet other embodiments include methods to detect a nerve's location within a region-of-interest of the muscular innervated tissue.

The method includes exposing portions of the region-of-interest with an energy source, the region-of-interest being in view of a camera; collecting signals arising from the exposed portions from an electromyleograph in signal communication with the innervated tissue exposed to the energy source; measuring the strength of the collected signals; mapping the measured signals within the region-of-interest onto an image of the region-of-interest captured by the camera, and correlating the mapped signals within the image of region-of-interest. The particular embodiments provide for exposing the region-of-interest with an energy source having at least one of infra red light, visible light, and ultraviolet light, and that mapping the measured signals within the region-of-interest includes Cartesian, radial, circular, and polar forms of mapping. Correlation of the mapped signals includes signal strength values that are overlaid upon or with the portions of the region-of-interest. Other particular embodiments provide that mapped signal correlation includes overlaying representations of the signal strength values with the portions of the region-of-interest. The overlaid representations may include visual-coding the signal strength values with the portions of the region-of-interest.

The particular embodiments also provide for a nerve response mapping system configured to detect a nerve or set of nerves associated with innervated tissue, including muscular tissue, located within a surgical region-of-interest of an organ or tissue region. The nerve response mapping system may also estimate, predict, or otherwise classify the nerve activity of degree of functionality by employing a means for exposing portions of the surgical region-of-interest with either a light energy source or an acoustic source, a means for obtaining an image of the region-of-interest, an electromyleograph in signal communication with the innervated tissue exposed to the light energy source or the acoustic energy source to generate signals arising from the exposed portions, a means for measuring the strength of the collected signals, a means for mapping the measured signals within the image of the region-of-interest, and a means for correlating the mapped signals with the image of the region-of-interest.

Alternate embodiments of the nerve response mapping system include that the energy source includes at least one of an infra red light, a visible light, an ultraviolet light, and an acoustic energy, and the means for mapping the measured signals within the region of interest includes a computer having a display of the region of interest configured to present representations of values of the collected signals with the portions at least one of a Cartesian, radial, circular, and polar map of the exposed portions overlaid onto the display, and the means for correlating the measured signals within the region of interest includes the computer system configured to present visual coding of the signal strength values with the portions of the region-of-interest.

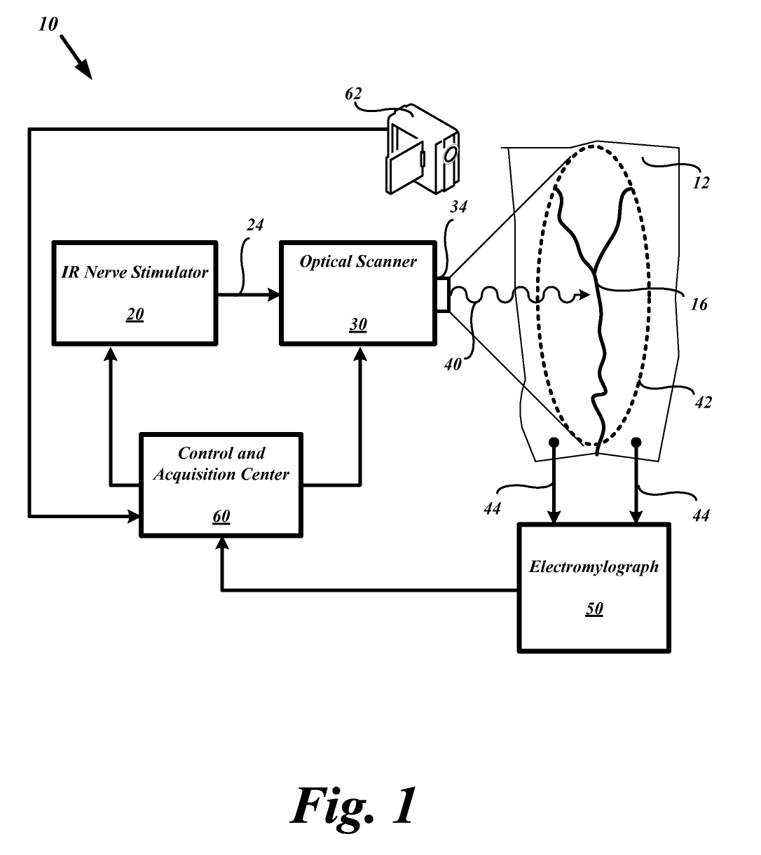

Yet other alternate embodiments of the nerve response mapping system include that the energy source may be housed in a wedge shaped enclosure that is configured for insertion between a nerve layer and a muscle layer of the region of interest that may be subjected to surgical procedures. From the wedge shaped housing, energies derived from infrared, ultraviolet, and/or acoustic energy are directed to the nerve layer for stimulation of a single nerve, a nerve branch, or nerve branches, and/or bundle of nerves. The direction of the light may be expansive over a substantial portion of the region of interest, or in a incremental manner by scanning in a series of raster like lines having scanning increments of light or acoustic cells definable in a Cartesian plane to obtain higher resolution signals more focused on smaller regions of the nerves, nerve branches, and/or nerve bundles. Via the electromyleograph in signal communication with the muscle layer, nerve activity from the energy stimulation is measured in the form of electromyleograph generated signals proportionately resulting from muscle movement generated from nerve activity communicated to the muscle layer as a result of the energy stimulation, either infrared, visible, ultraviolet, or acoustic delivered energy. Symbols or numerical values of electromyleograph generated signals proportionately resulting from fully active or functioning nerves to partially active or functioning nerves to virtually inactive or non-functioning nerves may then be plotted, in a map-like grid, as an overlay upon a displayed depiction of the surgical region of interest. The displayed depiction may include a “live” video image feed, a single screenshot image, and/or a graphical presentation of the region of interest. The symbols may be color coded or otherwise visually encoded to indicate differences in degrees of nerve activity as a function of nerve location stimulation. Patients undergoing radical prostatectomy bare bear the risk to of having nerve bundles along the periphery of the prostate severely compromised by encroaching tumor growth, and functionality significantly altered during surgical incisions made to remove the tumor growth or other compromised prostate regions. Optical nerve stimulation within the surgical field or region of interest is advantageous, as it does not create electrical artifacts in electromyleographs that commonly occurs by non-optical nerve stimulation processes. Thus, optical or photonic based EMS readings provide improved signal to noise ratios that exhibit high spatial selectivity, permitting a map of the nerve bundles in a region subject to surgical intervention. FIG. 1 depicts a functional block schematic of the Nerve Response Mapping (NRM) system 10. The NRM 10 includes an IR Nerve Stimulator 20 in light communication with an optical scanner 30 via a an optical fiber 24, an electromyleograph 50, and a Control and Acquisition center 60 in signal communication with the IR Nerve stimulator 20, the optical scanner 30, and the electromyleograph 50. Spatially accurate scans of the nerves relative to a body or anatomical location may employ locational techniques to correlate images to the anatomical locations or regions-of-interest undergoing examination and/or surgery. The locational techniques may include locally deployed coordinate based registration systems where camera coordinates and body coordinates are recorded, thereby providing a basis to correlate camera views to the anatomical theater or region being examined or undergoing surgery. Among the locational techniques employed may include the use of inertial reference units fully described in U.S. Patent Application Pub. No. US2007/0276247A1 to Chalana, et al., published Nov. 29, 2007, and filed Sep. 8, 2005 as U.S. patent application Ser. No. 11/222,360, herein incorporated by reference in its entirety. Other location and registration techniques may include those described in Lea, Jon et al, Registration and immobilization in robot-assisted surgery, Journal of Image Guided Surgery 1 (2), pp. 80-87, 1995, herein incorporated by reference in its entirety. Yet other location and registration techniques may include those described by McInerney, James, et al., Frameless Stereotaxy of the Brain, Mount Sinai Journal of Medicine, Vo. 67, No. 4, September, 2000, herein incorporated by reference in its entirety.

The NRM 10 also includes an optical window 34 of the optical scanner 30 that delivers light energy 40 received from the optical fiber 24 to a surgical field or surgical ROI 12 within an illuminated region 42 that exposes stimulating light to a nerve locus 16. The illuminated region 42 may be an even, near simultaneous illumination of a defined region, or progressively outlined in a near continuous or raster scan movement as a light bar is swept across the ROI 12. A camera image or series of images of the ROI 12 is captured by the camera 62 having a view of the ROI 12 and conveyed to the control and acquisition center 62. The camera 62 may be still camera or video, and the images conveyed may be analog and/or digital images.

The light energy 40 may be adjusted from infrared to ultraviolet energies to impart the optimal wavelength to promote nerve stimulation. When the light energy is other than infrared, the IR Nerve Stimulator 20 may be configured to be a Visible Light Nerve Stimulator 20 or an Ultraviolet Light Nerve Stimulator 20. The nerve locus 16 may include a single nerve, a plurality of nerves, or a bundle of interconnecting nerves. Nerve locus 16, upon being photo-stimulated from the optical scanner 30, causes muscle activity within the surgical field 12 in proportion to the native functionality of the nerve or nerves and generally in proportion to the light received. The muscle activity is measured by the EMS output delivered or conveyed through signal connectors 44 from the surgical ROI 12 to the electromyleograph 50. EMS signals are outputted to the control and acquisition center 60 to provide a nerve response map onto video or computer images of the ROI 12 within the illuminated region 40.

The nerve response map may extend beyond a single illuminated region 40 when the surgical ROI 12 is expanded beyond a single field of view. In the case of prostate surgery, the EMS indicates the activity of the cavernosal nerve of the penis. The same nerve may be stimulated on the exterior or anterior region of the prostate while the prostate is being surgically removed.

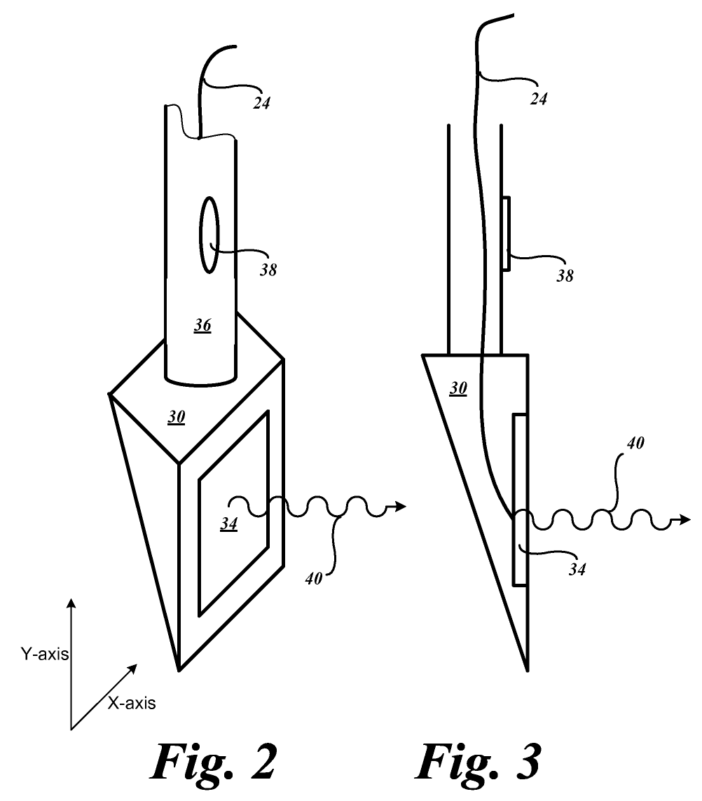

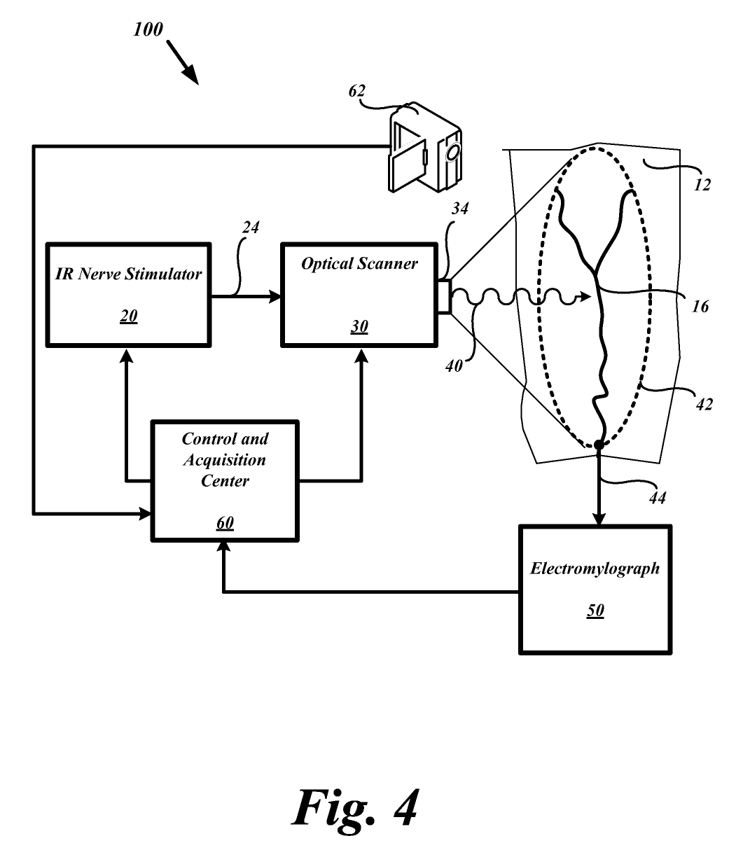

FIG. 2 depicts a perspective view of a particular embodiment of the optical scanner 30 used to stimulate nervous tissue. As shown the optical scanner 30 is wedge shaped. Connected to the scanner 30 is a pipe 36 that conveys the optical fiber 24 to the optical window 34 in which light energy 40 emanates. Externally mounted to the pipe 36 is a power indicating light source, for example a light emitting diode (LED) 38. The LED 38 provides a visual basis to confirm the position of the optical window 34 that delivers light energy 40 for neural stimulation. In alternate embodiments, the scanner 30 may be equipped with multiple windows 34 and with multiple LEDs 38 located at positions indicating which tissue is undergoing exposure from a given window 34 delivering nerve-stimulating energy 40. In yet other embodiments, the LED 38, may be replaced by an audible device, e.g., a speaker, to indicate the whereabouts of the nerves being stimulated within the light-irradiated tissue. Changes in audible pitch, pulsations, volume, or any combination of pitch, pulsations and/or volume may be configured to indicate the location and magnitude of neural stimulation. The LED 38 may be mounted on surgical eyeglasses or face shields within the field of view of a user. Similarly, the audio equivalent of the LED 38, for example, a miniature speaker, may be inserted close to the user's ear or within the auditory canal. FIG. 3 is a side view and cross-sectional schematic illustration of the optical scanner 30. The optical fiber 24 is shown routed internally within the pipe 36 and scanner 30 and connected with the optical window 34. As shown the optimal window 34 is presented on a single side only of the scanner 30. In other particular embodiments, more than one optical window 34 may be configured to operate independent from one another or in coordinated fashion so as to advantageously obtain neural map readings of multi-layer innervated tissue. For example, when the wedge shape scanner 30 is inserted between any two layers of an innervated organ, an activity map of a lower or bottom tissue layer may be obtained, and then with internal components (not shown) directing light from the optical fiber 24 to the optical window 34 located on the opposite side of the wedge, an activity map of the top or upper tissue layer may be obtained. The activity maps of either the bottom tissue layer or the top tissue layers may be overlaid upon a displayed depiction of the region-of-interest that may be subjected to surgical procedures. The displayed depiction may include “live” video feeds from the camera 62, or a single frame or screenshot image derived from images conveyed from the camera 62, or video or single frame graphical presentations derived from images conveyed from the camera 62. The LED 38 may be configured with direction indicators (not shown) to indicate which lower or upper tissue layer side is being activated. The direction indicators may be LEDs having different colors and/or intensities and/or different pulsations. Nerve Response Mapping 10 include the scanner 30 being in the form of a stereoscopic imaging device. The NRM 10 may be employed with laparoscopic procedures in which the laparoscope provides the image of the ROI 12 similar to that described for the camera 62. In other alternate embodiments the optical window 44 may be mounted on a moveable platform to provide progressive sweep like scanning across the ROI 12. FIG. 4 depicts an alternate functional block schematic embodiment of the Nerve Response Mapping system. The alternate NRM system 100 obtains photo induced nerve activity directly from a portion of the nerve. In system 100 a trunk of the nerve is tapped by signal probe 44. Nerve function of the tree like nerve locus 16 is measured as portions of the nerve locus 16 are exposed to infrared or other stimulating light energy 40.

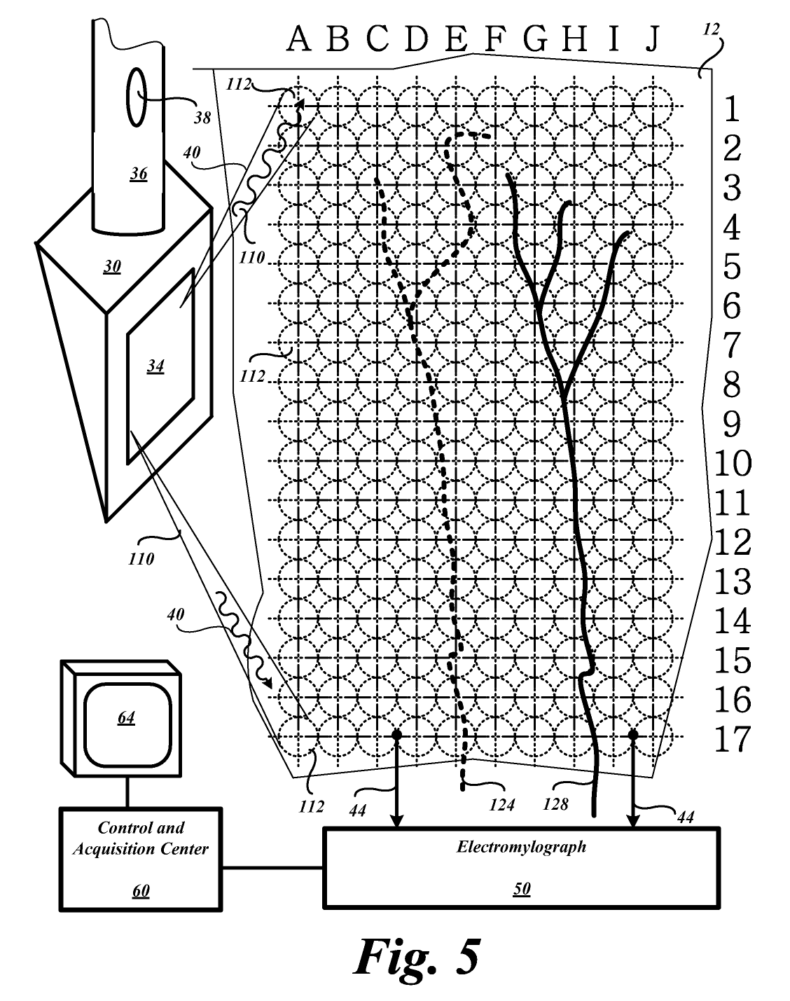

FIG. 5 depicts a scanning application of the Nerve Response Mapping system. Discrete scanning cones 110 having stimulating light energy 40 emanates from the optical window 34 from the scanner 30. The scanning cone 110 occupies forms a scanning cell 112 that incrementally moves in rows and/or columns within the surgical ROI 12. Each scanning cell 112 represents a portion of the surgical ROI 12. The surgical ROI 12 is thus divided up into multiple portions, and as illustrated, amenable to Cartesian grid mapping. The surgical ROI 12 includes a dotted two-branch nerve locus 124 and a solid line three-branch nerve locus 128. A grid labeled Cartesian designation references the incremental positions of the scanning cone 110 within the surgical ROI. As depicted, columns A-J represent X-axis positions, and rows 1-17 represent Y-axis positions, so that in this example, a 170-position mapping grid defines the surgical ROI 12. Thus the mapping grid incrementalizes the surgical ROI 12 into scanning increments or cells that can be described as map loci defined in Cartesian X,Y coordinates, here expressed in this illustration as column, row coordinates. In this figure, the two-branch nerve locus 124 is approximately located by the scanning cells A1 through E17, and the three-branch nerve locus 128 approximately occupies scanning cells F1 through J17.

The neural activity of nerve loci 124 and 128 within each branch or trunk of the nerve loci tree structures will be able to be mapped onto an image of the ROI 12 at a resolution defined by the size of the scanning cell 112. The size of the scanning cell 112 may be varied to accommodate different desired signal resolutions, so that the number of columns and rows may be varied beyond the 10 by 17 Cartesian coordinate combinations depicted in this figure. Generally, the smaller the scanning cell 112, the greater the resolution. EMS outputs through signal connectors 44 to the myleograph 50. The EMS signal outputs is relayed to the Control and Acquisition Center 60 and is overlaid onto the image of the surgical ROI 12 sent by the camera 62 and presented on monitor 64. A grid plot of the microvolt or millivolt or equivalent readings will guide the surgeon in where to, or where not to, cut depending on the nerve map overlay.

In alternate embodiments, the scan cells 112 may be defined by pinpoint lasers, infrared, visible, and/or ultraviolet lasers. At higher energies, for example red, green, blue, and ultraviolet energy, the power wattage may be progressively lowered at the lower light wavelengths so that light stimulation of the nerves is at a level that doesn't damage the nerves and/or surrounding tissue.

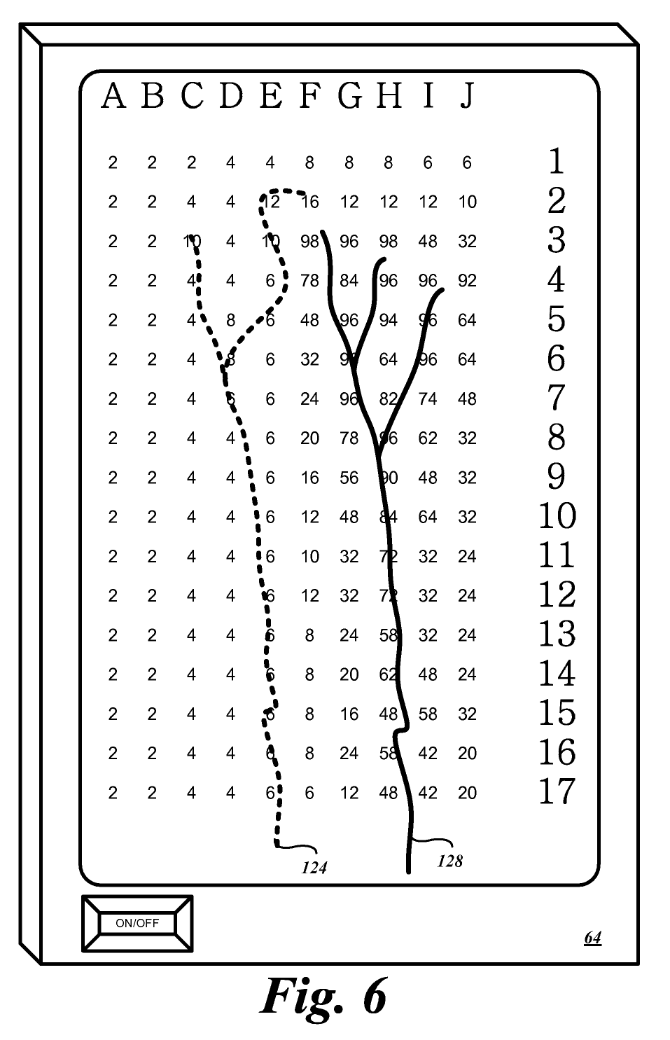

FIG. 6 depicts a nerve map overlay of myleograph signal output mapped onto the image the surgical ROI 12 depicted in FIG. 5. EMS values are overlaid on the scan cell grid. The two-branch nerve locus 124 has scan cell 112 includes scan readings ranging between 2 and 16 microvolts, with values along the branches and trunks ranging between 6 and 16 microvolts. The three-branch nerve locus 128 includes scan readings between 8 and 98 microvolts, with values along the branches and trunks ranging between 6 and 16 microvolts 42 and 98 microvolts. Presented with this information wherein the scan cells closer to the three-branch nerve locus 128 is approximately 7-fold the neural activity signal strength of the scan cells closer to the two branch nerve locus 124, a surgeon would conclude that cutting near the two-branch nerve locus 124 within cells A1-E17 presents less risk to the patient than cutting in the more neural active region defined by cells F1-J17. Representations of the signal strength obtained from the myleograph outputting signals arising from exposure to energy 40 may be overlaid onto the Cartesian grid portions or scan cells 112 instead of the numerical values depicted. The representations may visual coding of the numerical values, including color-coding. For example, the low activity nerve locus 124 may be overlaid with gray to light blue colors for scan cells 112 having values between 2 and 12, and shades of yellow to orange to bright red for high activity nerve locus 128 having values between 19 and 99. Other visual codings may employ degrees of cross hatching, stippling, or other forms of visual markings that serves to provide contrast to the viewing physician on where and how active a given nerve or nerve bundle is within the surgical ROI 12. FIGS. 5 and 6 depict scenarios in which adjacent nerves substantially occupy the sample tissue layer. In multi layer organs having different levels of innervated neuromuscular tissue, as in the case of prostate surgery, the difficulty facing the surgeon is in determining which layer of the prostate is internal or external to a given nerve bundle, and how active is the given nerve bundle. To determine muscular layer location relative to stacked nerve bundles that may present varying functional activities, the surgeon inserts the wedge shaped scanner 30 between any two layers of the prostate. The LED 38 would indicate which side of the prostate layer is being optically stimulated by energy 40. An anatomical and nerve activity map of the surgical ROI 12 similar to that presented in FIG. 6 is obtained for each nerve bundle of each layer from the raster like scanning presented in FIG. 5. The physician would then examine the contemporaneous acquired color-coding or other visual presentation to provide accurate positional and neural activity assessments from the display 64 for each nerve layer bundle to thereby be instantly informed of where to conduct surgery with the best outcome to the patient. While the particular embodiments for systems and methods have been illustrated and described for nerve response mapping from light energy sources, other maps may obtained there nerve activation by acoustic sources, including ultrasonic focused scans. Moreover, the nerve activity maps other than Cartesian generated may be developed, for example radial, circular, or polar map distributions may be sued. Accordingly, the scope of embodiments of the invention is not limited by the disclosure of the particular embodiments. Instead, embodiments of the invention should be determined entirely by reference to the claims that follow.

The embodiments of the invention in which an exclusive property or privilege is claimed are defined as follows:

1. A method to detect a nerve associated with innervated tissue located within a region-of-interest comprising:
exposing portions of nerves within the region-of-interest with to energy from an energy source, the region-of-interest being in view of a camera;
collecting signals arising from promulgated within the nerves, in response to exposure, the exposed portions format an electromyleograph in signal communication with the innervated tissue exposed to the energy source;
measuring the strength of the collected signals; and
mapping the measured signals within the region-of-interest onto an image of the region-of-interest captured by the camera; and
correlating the mapped signals within the image of region-of-interest.

2. The method of claim 1, wherein exposing the region-of-interest with the energy from an energy source includes at least one of the group consisting of infra red light, visible light, ultraviolet light, and acoustic energy.

3. The method of claim 1, wherein mapping the measured signals within the region-of-interest includes mapping according to at least one of the coordinates group consisting of Cartesian, radial, circular, and polar.

4. The method of claim 3, wherein correlating the mapped mapping signals includes signal strength values overlaid with the portions of the region-of-interest.

5. The method of claim 4, wherein correlating the mapped signals includes overlaying representations of the signal strength values at nerve locations with the portions ofin the images of the region-of-interest.

6. The method of claim 5, wherein overlaying representations include overlaying visual-coding of the signal strength values with the portions ofin images of the region-of-interest.

7. The method of claim 5, wherein measuring the strength of the collected signals is used to generate conveyed by at least one of a group of indicator signals consisting of visual, auditory, and graphical presentation.

8. A system to detect a nerve associated with innervated tissue located within a region-of-interest comprising:
means for exposing nerves portions of within the region-of-interest with to energy emanating from an energy source;
means for obtaining an image of the region-of-interest;
an electromyleograph in signal communication with the innervated tissue exposed to the energy source to generate to collect signals arising promulgated within the nerves from the exposed portions in response to exposure to the energy;
means for measuring the strength of the collected signals; and
means for mapping the measured signals within the image of the region-of-interest; and
means for correlating the mapped signals with the image of the region-of-interest.

9. The system of claim 8, wherein the energy source includes at least one of an infra red light, a visible light, an ultraviolet light, and an acoustic energy.

10. The system of claim 8, wherein means for mapping the measured signals within the region of interest includes a computer having a display an image of the region of interest and configured to present representations of values of strengths of the collected signals with the portions on the image by at least one of a group of coordinate systems including Cartesian, radial, circular, and polar map of the exposed portions overlaid onto the display.

11. The system of claim 10, wherein means for correlating mapping the measured signals within the region of interest includes the computer system configured to present visual coding of the signal strength values with the portions of the region-of-interest.

12. The system of claim 9, wherein the energy source is house in a wedge wedge-shaped enclosure.

13. The system of claim 12, wherein the wedge wedge-shaped enclosure is configured for insertion between innervated layers of the region of interest, including a nerve layer and a muscle layer of the region of interest.

14. The system of claim 13, wherein energy emanating from the energy source of the wedge wedge-shaped enclosure exposes the nerve layer to includes at least one of the group consisting of infrared light, the visible light, the ultraviolet light, and the acoustic energy.

15. The system of claim 14, wherein means for obtaining an image of the region-of-interest the energy source is conveyed to the region of interest into includes scanning by increments definable in a Cartesian plane.

16. The system of claim 15, wherein the measured strengths of signals promulgated within the nerves measured signals from nerves responding to the stimulating energy within the scanning increments are representable by at least one of symbolic depictions and numerical values that are overlaid upon a displayed depiction of the region of interest.

17. The system of claim 13, wherein the wedge wedge-shaped enclosure includes a light source to indicate which layer of the innervated layers is being stimulated.

18. The system of claim 17, wherein the light source includes at least one light emitting diode.

19. The system of claim 18, wherein the at least one light emitting diode includes directional indicators to denote which layer of the innervated layer is being stimulated.

20. The system of claim 19, wherein the directional indicators may denote which layer of the innervated layer is being stimulated by differences in colors, intensities, and pulsation frequency.
